(19) United States
(12) Patent Application Publication
(10) Pub. No.: US 2013/0023002 A1
(43) Pub. Date: Jan. 24, 2013

(54) USE OF A SUBSTRATE IN A METHOD FOR MEASURING THE ACTIVITY OF AVAILABLE PROTEOLYTIC ENZYMES

(71) Applicants: Gérard QUENTIN, Asnieres sur Seine (FR); Audrey CARLO, Asnieres sur Seine (FR); Loïse BOULANGER, Asnieres sur Seine (FR)

(72) Inventors: Gérard QUENTIN, Asnieres sur Seine (FR); Audrey CARLO, Asnieres sur Seine (FR); Loïse BOULANGER, Asnieres sur Seine (FR)

(21) Appl. No.: 13/553,932

(22) Filed: Jul. 20, 2012

(30) Foreign Application Priority Data
Jul. 22, 2011 (FR) 1156714

(51) Int. Cl. C12Q 1/56

(52) U.S. Cl. 435/13

(57) ABSTRACT

The disclosure relates to the use of a proteolytic enzyme substrate of general formula: Q¹-Xaa²-Xaa¹-rhodamine₁₁₀-Q², in which: Xaa¹ and Xaa² are amino acids and Q² is a group comprising an alkyl group; so as to be able to allow, in a blood sample containing a glycosaminoglycan, said glycosaminoglycan to inhibit the coagulation of said blood sample, via which the anticoagulant capacity of the glycosaminoglycan is not disrupted.

The present invention relates to the use of a proteolytic enzyme substrate, in particular in a method for measuring the activity of proteolytic enzymes in a blood sample. The invention also relates to a measuring kit which makes it possible to implement said method. More particularly, the method aims to measure, in a blood sample belonging to a patient treated with an anticoagulant of the glycosaminoglycan family, the activity of the enzymes responsible for hemostasis. This physiological process, which aims to interrupt bleeding, involves a cascade of enzymes and of coagulation factors. The anticoagulant is generally a molecule of the glycosaminoglycan family and more particularly a heparin. A distinction will be made between high-molecular-weight heparins termed: “unfractionated heparins” or abbreviated to UFH, and heparins termed: “low-molecular-weight heparins” or abbreviated to LMWH. The various heparins are administered to patients according to their pathological condition.

Usually, measuring methods aim to test the activity of enzymes, for example thrombin or plasmin. The enzyme substrates used in these measuring methods are then formed from a synthetic peptide consisting of two or three amino acids having an affinity for the enzyme and of a fluorophore group bonded to said peptide. The enzyme is then capable of cleaving the substrate between the fluorophore and the peptide, said fluorophore changing state and then being capable of providing a detectable fluorescent signal. Thus, the substrate is also referred to as a tracer reagent.

Reference may be made to document EP 1 833 982 which describes, for example, a thrombin-specific enzyme substrate which has a tripeptide coupled to 7-amino-4-methylcoumarin, commonly abbreviated to AMC. It also describes a plasmin-specific enzyme substrate which has a bis-dipeptide coupled to rhodamine₁₁₀.

According to the usual methods for measuring the activity of proteolytic enzymes, a blood sample, of blood or plasma, is provided and brought into contact with an activator, for example tissue factor, which will make it possible to activate the production of the enzyme. Next, the substrate, or tracer, is provided with an initiator, for example calcium in ionic form. The blood sample and the activator are then brought into contact with the substrate and the initiator in the same medium in which, on the one hand, the coagulation reactions and, on the other hand, the reaction of the enzyme with the substrate will take place. The initiator will make it possible to initiate the coagulation process, while the activator induces the production of the enzyme.

The use of a tracer including rhodamine₁₁₀ in these measuring methods has proved to be advantageous, since this fluorophore exhibits excitation and emission wavelengths which are shifted with respect to the 7-amino-4-methylcoumarin normally used for this type of measurement, and which do not interfere with the hemoglobin possibly present in the blood sample.

However, differences in activity measurement have been encountered between substrates including rhodamine₁₁₀ and the usual substrates including 7-amino-4-methylcoumarin, according to various blood samples.

Thus, a problem which arises and which the present invention aims to solve is that of providing a tracer substrate for proteolytic enzymes which includes rhodamine₁₁₀ and which makes it possible to carry out coherent activity measurements according to various blood samples, when said blood samples contain an anticoagulant of glycosaminoglycan type.

With this objective, the present invention proposes the use of a proteolytic enzyme substrate of general formula: Q¹-Xaa²-Xaa¹-rhodamine₁₁₀-Q², in which: Xaa¹ and Xaa² are amino acids; and Q² is a group comprising an alkyl group; so as to be able to allow, in a blood sample containing a glycosaminoglycan, said glycosaminoglycan to inhibit the coagulation of said blood sample, and consequently, to modulate and in particular reduce the activity of certain proteolytic enzymes. Thus, the anticoagulant capacity of the glycosaminoglycan is not disrupted.

Thus, one characteristic of the invention lies in the updating of the neutralizing capacity of certain substrates including rhodamine₁₁₀ and in the identification of a substrate with a particular structure which makes it possible precisely not to neutralize the glycosaminoglycans contained in the various blood samples. In that way, the measurement of the activity of proteolytic enzymes can be carried out on blood samples from patients treated with anticoagulants while taking into account the effects of the treatment. This makes it possible in particular to modulate them.

It has in fact been possible to note that the usual substrates including 7-amino-4-methylcoumarin, as fluorophore, have no effect with respect to the various glycosaminoglycans commonly used as anticoagulants.

Consequently, it is now possible to use a proteolytic enzyme substrate in accordance with the invention, for optically measuring the apparent enzymatic activity of the blood sample from patients treated with anticoagulants, in an excitation and emission wavelength range which is advantageous in particular with respect to hemoglobin.

According to one particularly advantageous embodiment of the invention, Q¹ is H; or the R¹O—(C═O)—CH₂—CO group in which R¹ comprises an alkyl or aryl group; or the R²—Xaa³ group in which Xaa³ is an amino acid and R² is a protective group or H. In that way, an enzyme substrate exhibiting good solubility is obtained, and consequently a homogeneous reactive medium. The fluorescence measurements are thus reproducible.

Advantageously, Xaa¹ is a basic amino acid, for example arginine. In that way, the substrate offers perfect recognition of the enzyme.

According to one particular embodiment of the invention, Q¹ is the R²—Xaa³ group, Xaa² and Xaa³ being respectively glycine, so as to confer on said substrate a low affinity with respect to the proteolytic enzymes. The substrate with such a structure makes it possible to more easily measure the monitoring of the appearance of an enzymatic activity, more commonly called “generation”. This is because in fact, in the case of measurements of this type, it is important to take into account the whole of the cascade of enzymatic reactions and not to disrupt their kinetics. Since the substrate has a lower affinity with respect to the enzymes, the variation in the concentration of enzymes mobilized by the hydrolysis of the substrate will therefore be negligible and will have a very small impact on the rate of the other enzymatic reactions of the cascade. Although the reaction of the substrate which is the subject of the invention and of the enzyme under consideration has a relatively fragile rate constant, the fluorescent signal will nevertheless be considerable and detectable since rhodamine₁₁₀ exhibits a large amount of fluorescence.

According to another particular embodiment, in which Q¹ is the R¹O—(C═O)—CH₂—CO group, Xaa² being valine and R¹ the ethyl group, a low affinity with respect to proteolytic enzymes is conferred on said substrate. Consequently, the appearance of an enzymatic activity is also measured without disrupting the kinetics of the enzymatic reaction cascade.

According to another variant of embodiment of the invention, Q² comprises a —(C═O)ₘ—(O)ₙ— group linked to rhodamine₁₁₀, in which n=0 or 1, and m=0 or 1. In that way, the substrate is easier to produce. Indeed, by virtue of the —(C═O)ₘ—(O)ₙ— group, the attachment of the group comprising the alkyl group to rhodamine₁₁₀ is facilitated. Advantageously, n=0 and m=1, and the group is simply a carbonyl function. For example, Q² is the acetyl group or else the 3-methylbutanoyl group.

According to another subject, the present invention proposes a method for measuring the activity of proteolytic enzymes of a blood sample, said method being of the type according to which: a blood sample which contains proteolytic enzymes and which may contain a glycosaminoglycan is provided; a proteolytic enzyme substrate capable of providing a signal when said proteolytic enzymes react with said substrate is provided; said blood sample is brought into contact with said proteolytic enzyme substrate, so as to allow said proteolytic enzymes to react with said substrate in order to provide a signal representative of the activity of the proteolytic enzymes in said sample. According to the invention, a proteolytic enzyme substrate of general formula: Q¹-Xaa²-Xaa¹-rhodamine₁₁₀-Q², in which: Xaa¹ and Xaa² are amino acids; and Q² is a group comprising an alkyl group, is used so as to be able both to allow the glycosaminoglycan to inhibit the coagulation of said blood sample and to measure the activity of the proteolytic enzymes, via which the signal measured is representative of the activity of only the proteolytic enzymes capable of bringing about the coagulation. The proteolytic enzyme substrate used includes of course all the abovementioned variants and embodiments.

Preferentially, an activating reagent which makes it possible to induce the generation of proteolytic enzymes is also provided, and said activating reagent is brought into contact with said blood sample and said proteolytic enzyme substrate. As soon as the activating reagent comes into contact with the blood sample, it causes the generation of the proteolytic enzymes. For example, said activating reagent is chosen from tissue factor, phospholipids, thromboplastin, kaolin, ellagic acid, collagen, adenosine diphosphate, arachidonic acid, a thrombin receptor-activating peptide, or a combination thereof. Advantageously, an activator which is capable of acting a long way upstream in the coagulation and fibrinolysis enzymatic reaction cascade will be chosen, in particular when the measurement is aimed at monitoring the appearance of an enzymatic activity.

In addition, an initiating reagent for initiating the proteolytic enzyme reactions is provided, and said initiating reagent is brought into contact with said blood sample and said proteolytic enzyme substrate. In that way, the initiator will make it possible to initiate the coagulation process, while the activator brings about the generation of the enzymes. Advantageously, said initiating reagent is calcium, and it is provided, for example, in the form of calcium citrate.

According to one embodiment of the measuring method in accordance with the invention, said blood sample is brought into contact with said proteolytic enzyme substrate at a substrate concentration of between 50×10⁻⁶ and 1000×10⁻⁶ mol.L⁻¹, for example between 300×10⁻⁶ and 500×10⁻⁶ mol.L⁻¹. In that way, a sufficiently strong optical signal is obtained, making it possible to measure the enzymatic activity.

According to yet another subject, the present invention proposes a kit for measuring the activity of proteolytic enzymes of a blood sample, for implementing the measuring method described above. According to the invention, the kit comprises: an activating reagent for inducing the generation of proteolytic enzymes; a proteolytic enzyme substrate of general formula Q¹-Xaa²-Xaa¹-rhodamine₁₁₀-Q², in which: Xaa¹ and Xaa² are amino acids; and Q² is a group comprising an alkyl group; and an initiating reagent for initiating the enzymatic reactions. The proteolytic enzyme substrate used includes all the abovementioned variants and embodiments.

According to a first embodiment variant of the kit, said proteolytic enzyme substrate is mixed with said initiating reagent, in the same container, whereas the activating reagent is contained in another container. The enzyme substrate is then, for example, at a concentration of between 100×10⁻⁶ mol.L⁻¹ and 500×10⁻⁶ mol.L⁻¹, while the initiating reagent is at a concentration of between 10×10⁻³ mol.L⁻¹ and 20×10⁻³ mol.L⁻¹. The activating reagent is itself at a concentration of between, for example, 0.5×10⁻¹² and 2×10⁻¹² mol.L⁻¹.

According to a second variant, said activating reagent is mixed with said initiating reagent in the same container, whereas the enzyme substrate is contained alone in another container. This second embodiment variant makes it possible in particular not to disrupt the stability of the substrate.

When the kit which is the subject of the invention is used, firstly 40 μL of a blood sample for example, and secondly 10 μL of enzyme substrate, alone or mixed with the initiating reagent, and 10 μL of the activating reagent, respectively mixed with the initiating reagent or alone, are for example provided. These sample and reagent volumes are of course indications, and are adjusted according to the measuring devices and also to the types of measurement. They can just as easily be multiples thereof, or else other volumes, for example 30 μL/15 μL/15 μL.

Other particularities and advantages of the invention will emerge on reading the description provided hereinafter of a particular embodiment of the invention, given by way of nonlimiting indication, with reference to the appended drawings in which:

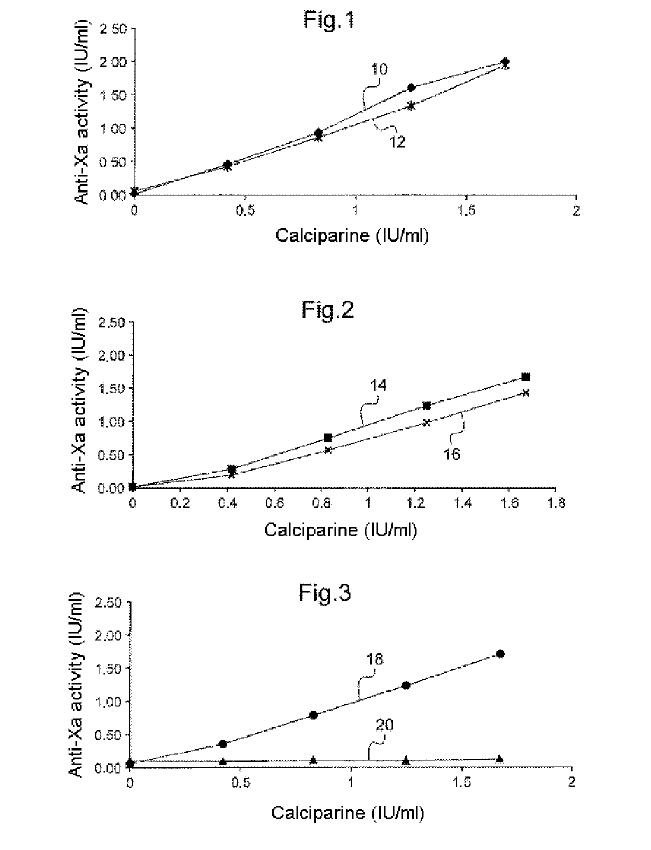

FIG. 1 is a graph showing enzymatic activities as a function of a heparin concentration according to a first example of implementation of an evaluation method;

FIG. 2 is a graph showing enzymatic activities as a function of a heparin concentration according to a second example of implementation of an evaluation method;

FIG. 3 is a graph showing enzymatic activities as a function of a heparin concentration according to a third example of implementation of an evaluation method;

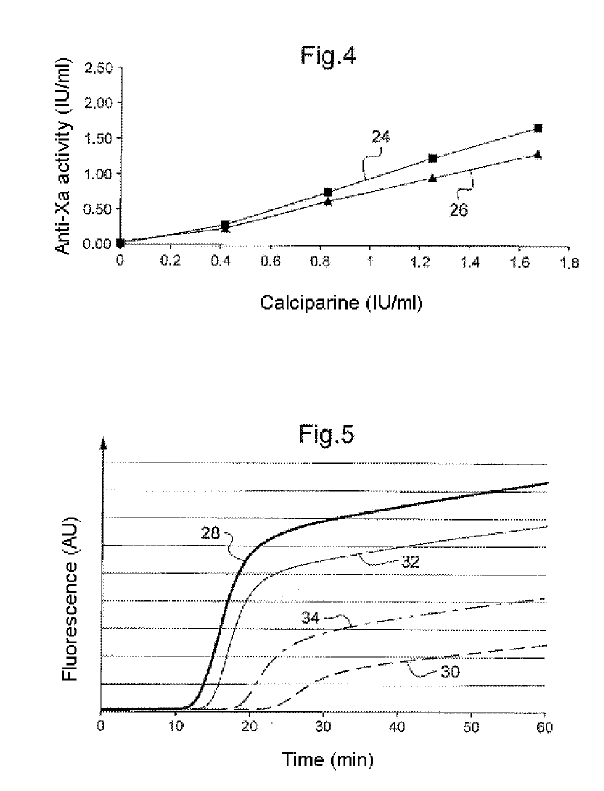

FIG. 4 is a graph showing enzymatic activities as a function of a heparin concentration according to a fourth example of implementation of an evaluation method; and

FIG. 5 is a graph showing the strength of a fluorescent signal as a function of time according to the measuring method in accordance with the invention, with an unfractionated heparin at various concentrations.

The object of the invention is in particular to use proteolytic enzyme substrates which make it possible to measure the activity of the enzymes in the blood sample from a patient, without disrupting the medium during the measurement and in particular without interfering with heparin when the patient is taking anticoagulants.

Substrates of which the fluorophore is 7-amino-4-methylcoumarin, commonly referred to as AMC, are recognized as having no neutralizing effect with respect to glycosaminoglycans and in particular to heparin.

In a first part of the detailed description, firstly it will be explained how the neutralization of these glycosaminoglycans contained in a blood sample is evaluated, and secondly, examples will be given of a proteolytic enzyme substrate of which the fluorophore is rhodamine, of general formula: Q¹-Xaa²-Xaa¹-rhodamine₁₁₀-Q², and which do not interfere with glycosaminoglycans. This fluorophore is selected since it exhibits excitation and emission wavelengths which are shifted with respect to 7-amino-4-methylcoumarin and which do not interfere with the hemoglobin of the blood sample.

It will be observed that one of the merits of the invention has been to bring to light the effect of certain proteolytic enzyme substrates, when their fluorophore is rhodamine₁₁₀, on the anticoagulant capacity of glycosaminoglycans. In addition, this principle of innocuousness of the substrates which are the subject of the invention, with respect to two types of heparins, unfractionated heparins, or UFHs, and low-molecular-weight heparins, or LMWHs, will be verified.

Next, in a second part of the description, an enzyme substrate identified in the first part will be applied in a measuring method in accordance with the invention, in which the heparins are insensitive to its presence in the medium, and where they make it possible to measure the activity of the enzymes.

Before referring to the diagrams of the figures, the evaluation method used to quantify the neutralizing effect of the proteolytic enzyme substrates with respect to the glycosaminoglycans in a blood sample, and in particular a plasma, will be described. This evaluation method makes it possible to use both unfractionated heparins and low-molecular-weight heparins. In addition, the method endeavors to test the activity of factor Xa, located at the crossroads of two enzymatic cascades resulting in the formation of thrombin. Moreover, antithrombin, which is a natural inhibitor of thrombin generation, controls the reaction. As soon as it appears, factor Xa couples to its natural substrate, prothrombin, so as to form thrombin. The heparin contained in the sample forms, itself, a complex with antithrombin, which complex promotes the inhibition of factor Xa.

Thus, a chromogenic substrate based on para-nitroaniline, which factor Xa hydrolyzes, is used and the release of the para-nitroaniline, which can be measured optically, is then inversely proportional to the concentration of heparin present in the medium. A measurement of the anti-Xa activity is thus deduced therefrom.

Reference will be made to the diagram of FIG. 1, for analyzing the results of a first reference example implementing the abovementioned evaluation method.

In this first example, the heparin used is an unfractionated calcium heparin sold under the brand name: “Calciparine®”. According to the evaluation method, the anti-Xa activity is recorded and transferred onto the y-axis, as a function of the heparin concentration of the medium. The heparin concentrations vary between 0 and 1.67 IU/ml and are relatively close to the therapeutic concentrations. The first inclined curve 10 corresponds to a first series of measurements wherein physiological saline is added to the blood sample in addition to the para-nitroaniline-based substrate. The second inclined curve 12 corresponds to a second series of measurements wherein a usual substrate, of which the fluorophore is 7-amino-4-methylcoumarin, and the synthetic formula of which is Z-GGR-AMC, in which R corresponds to arginine, G corresponds to glycine and wherein Z is a protective group, is added to the blood sample. This tracer substrate is commonly used in methods for measuring enzymatic activity.

Thus, the curves 10, 12 illustrate the variations in anti-Xa activity as a function of the heparin concentration of the medium.

Since physiological saline has by nature no effect on the anticoagulant capacity of heparin and since the two curves are substantially the same, it is quite naturally and qualitatively deduced therefrom that the Z-GGR-AMC substrate has no notable effect with respect to heparin.

More specifically, the percentage neutralization by the substrate is determined as being the ratio of the difference in the variation in anti-Xa activity in the medium containing the physiological saline and in the medium containing the substrate, between two heparin concentrations, and only the variation in activity in the medium containing the physiological saline, between the same two heparin concentrations, this ratio being multiplied by 100 in order to obtain the result as percentage neutralization.

The percentage neutralization can also be written more compactly: 100×(1−ΔA₁/ΔA₂), wherein ΔA₁ corresponds to the variation in activity in the presence of substrate and ΔA₂ to the variation in the presence of physiological saline. Thus, the parallel measurement of the variation in the presence of physiological saline makes it possible to establish the reference in each series of measurements.

In this first example of implementation, the percentage neutralization of the substrate of synthetic formula Z-GGR-AMC varies between 5% and 20%. The impact of the substrate on the anticoagulant effect of heparin is then sufficiently low for the activity measurement to be revealing, as will be explained hereinafter in greater detail.

Reference will now be made to the diagram of FIG. 2, showing the results of a second example of implementation of the abovementioned evaluation method, in which the two curves 14, 16 are comparable to those of the first example. The first curve 14 corresponds to a first series of measurements carried out under conditions similar to those of the first example, in which physiological saline is added to the blood sample. As for the second curve 16, it corresponds to a second series of measurements wherein a proteolytic enzyme substrate in accordance with the invention of formula: Q¹-Xaa²-Xaa¹-rhodamine₁₁₀-Q², in which, according to a first variant V(1), Q¹ is the R¹O—(C═O)—CH₂—CO group, Xaa¹ being arginine, Xaa² valine and R¹ the ethyl group, while Q² is the acetyl group, is added to the blood sample. The substrate which is the subject of this first variant is written synthetically as: EtM-VR-Rhod₁₁₀-Ac.

This proteolytic enzyme substrate obviously plays no role here in the evaluation method as tracer. This second example has no objective other than to show that this substrate has very little impact on the anticoagulant effect of heparin.

The heparin used is also Calciparine®, and it is observed that the percentage neutralization obtained according to the formula given above: 100×(1−ΔA₁/ΔA₂), varies between 15% and 30% as a function of heparin concentrations. As will be explained in the remainder of the description, this enzyme substrate is a good candidate for the measuring method according to the invention, since it makes it possible to measure the enzymatic activity without affecting the anticoagulant capacity of heparin.

Reference will be made to the diagram of FIG. 3, illustrating the results of a third example of implementation of the evaluation method. A first curve 18 corresponding to a first series of measurements is produced under conditions similar to the preceding examples, in which physiological saline is added to the blood sample.

A second series of measurements is then carried out, wherein a proteolytic enzyme substrate, not in accordance with the invention, but of synthetic formula R²-GGR-Rhod₁₁₀-R, in which R is arginine and G glycine, while R² is a protective group, is added to the blood sample. As can be observed, the curve 20 is substantially horizontal and the percentage heparin neutralization by this substrate, obtained according to the formula 100×(1−ΔA₁/ΔA₂), is close to 100%. Under these conditions, the substrate in question is not a good candidate for the measuring method that will be described hereinafter. This third example is of course a counterexample.

Reference will now be made to the diagram of FIG. 4, showing the results of a fourth example of implementation of the abovementioned evaluation method, in which the two curves 24, 26 are comparable to those of the first and second examples. The first curve 24 corresponds to a first series of measurements carried out under conditions similar to those of the first example, in which physiological saline is added to the blood sample. As for the second curve 26, it corresponds to a second series of measurements wherein a proteolytic enzyme substrate in accordance with the invention, according to a second variant V(2) of synthetic formula R²-GGR-Rhod₁₁₀-3-methylbutanoyl, in which R is arginine and G glycine, while R² is a protective group, is added to the blood sample. It will be observed that this substrate differs from that of the third example only in terms of the -3-methylbutanoyl substituent.

This proteolytic enzyme substrate also plays no role in the evaluation method as a tracer. This fourth example also shows that this substrate has very little impact on the anticoagulant effect of heparin.

The heparin used is also Calciparine®, and it is observed that the percentage neutralization obtained according to the formula given above: 100×(1−ΔA₁/ΔA₂), varies between 20% and 25% as a function of the heparin concentrations. As will be explained in the remainder of the description, this enzyme substrate is also a good candidate for the measuring method according to the invention, since it makes it possible to measure the enzymatic activity without affecting the anticoagulant capacity of heparin.

According to a third embodiment variant, which is not illustrated, still according to the same protocol as that of the above examples, the substrate has the synthetic formula R²-GGR-Rhod₁₁₀-Ac in which R is arginine and G glycine, while R² is a protective group. It differs from the preceding one only in terms of the acetyl substituent. The percentage neutralization then varies between 12% and 18%. This substrate is thus an excellent candidate for the measuring method according to the invention.

Still in this first part of the description, the abovementioned evaluation method was implemented with a low-molecular-weight heparin, Fragmine®.

The results of the percentage neutralization of this heparin for the three substrate variants described above, V(1), V(2) and V(3), have been reported in the table below.

TABLE I

| [IU/ml] | V(1) | V(2) | V(3) |
|---|---|---|---|
| 0.42 | 10% | 5% | 1% |
| 0.83 | 12% | 11% | 5% |
| 1.25 | 13% | 8% | 7% |
| 1.67 | 15% | 4% | 7% |

This table I shows that the enzyme substrate in accordance with the invention, according to the three embodiment variants previously described, exhibits a very low neutralizing capacity with a low-molecular-weight heparin.

Thus, the present invention also relates to a method for measuring the activity of proteolytic enzymes of a blood sample, which makes use of an enzyme substrate of general formula: Q¹-Xaa²-Xaa¹-rhodamine₁₁₀-Q², in which: Xaa¹ and Xaa² are amino acids; and Q² is a group comprising an alkyl group. By virtue of this enzyme substrate, the activity of an enzyme is measured without inhibiting the anticoagulant capacity of the heparins in a blood sample, and this is, moreover, the advantage of the use of the substrates which are the subject of the invention.

According to one embodiment of the measuring method, the enzymatic activity monitored is that of thrombin generation. The principle lies in the monitoring of the measurement of a fluorescent signal after the enzyme substrate has been brought into contact with a blood sample containing heparin, and the reaction conditions allow the thrombin to cleave the substrate between the peptide part and the fluorophore, in this case rhodamine₁₁₀.

In order to implement the method, a blood sample of 40 μL containing heparin is first of all introduced into a microcuvette. 10 μL of the enzyme substrate in accordance with the invention mixed with an initiating reagent, and 10 μL of activating reagent are added thereto. The initiating reagent is calcium citrate and it has a concentration of 16.7×10⁻³ mol.L⁻¹. The activating reagent is tissue factor at a concentration of 10⁻¹² mol.L⁻¹.

Next, an excitation signal having a predetermined wavelength is emitted through the microcuvette and the response to the excitation signal is recorded optically over time. Thus, the greater the amount of thrombin generated, the greater the signal response.

According to one particular embodiment, the enzyme substrate chosen is that corresponding to the embodiment variant V(3) of the first part of the detailed description, and of synthetic substrate formula is of synthetic formula R²⁻GGR-Rhod₁₁₀-Ac, in which R is arginine and G glycine, while R² is a protective group and Ac the acetyl group. In addition, the method is in this case implemented with an unfractionated heparin. It could very well be implemented with a low-molecular-weight heparin, given the above evaluation results.

The results are reported in the graph of FIG. 5. It illustrates a series of experiments using an unfractionated heparin at increasing concentrations of 0.42 IU/ml, 0.83 IU/ml, 1.25 IU/ml and 1.67 IU/ml. The strength of the excitation-signal response is reported on the y-axis, while the time scale is represented on the x-axis.

Thus, it is observed that the upper curve 28 corresponds to the heparin concentration of 0.42 IU/ml, that the lower curve corresponds to the heparin concentration of 1.67 IU/ml, and that the two intermediate curves, 32, 34, correspond respectively to the heparin concentrations of 0.83 IU/ml and 1.25 IU/ml.

In that way, it is clearly demonstrated that the substrate chosen does not interfere with the anticoagulant effect of heparin, since the amount of thrombin detected decreases substantially linearly with the increase in the heparin concentrations. Thus, by virtue of the substrate selected, it is possible to measure the thrombin activity in the blood sample from a patient treated with anticoagulants, without this substrate interfering with the effect of this anticoagulant on the activity of the proteolytic enzymes.

The present invention also relates to a kit for measuring the activity of proteolytic enzymes of a blood sample, for implementing the measuring method described above.

According to the invention, the kit comprises a substrate according to one of the variants V(1) to V(3). Of course, it could comprise a substrate according to another variant defined by the abovementioned general formula. The proteolytic enzyme substrate, at a concentration of 300×10⁻⁶ mol.L⁻¹, is then mixed with calcium citrate at a concentration of 16.7×10⁻³ mol.L⁻¹, in a first container. This mixture forms a first reagent. Tissue factor at a concentration of 10⁻¹² mol.L⁻¹ is contained in another container and constitutes the second reagent.

These two reagents are then capable of being used in a microcuvette, with the blood sample, at the time the enzymatic activity is measured.

1. A method of inhibiting, in a blood sample containing a glycosaminoglycan, coagulation of said blood sample, via which the anticoagulant capacity of the glycosaminoglycan is not disrupted, comprising applying a proteolytic enzyme substrate of general formula: Q¹-Xaa²-Xaa¹-rhodamine₁₁₀-Q², in which:
Xaa¹ and Xaa² are amino acids; and
Q² is a group comprising an alkyl group.

2. The method of claim 1, wherein in the proteolytic enzyme substrate Q¹ is:
H; or
the R¹O—(C═O)—CH₂—CO group in which R¹ comprises an alkyl or aryl group; or
the R²—Xaa³ group in which Xaa³ is an amino acid and R² a protective group or H.

3. The method of claim 2, wherein in the proteolytic enzyme substrate 2, Xaa¹ is a basic amino acid.

4. The method of claim 3, wherein in the proteolytic enzyme substrate Xaa¹ is arginine.

5. The method of claim 4, wherein in the proteolytic enzyme substrate Q¹ is the R²—Xaa³ group, Xaa² and Xaa³ being respectively glycine, so as to confer on said substrate a low affinity with respect to proteolytic enzymes.

6. The method of claim 4, wherein in the proteolytic enzyme substrate Q¹ is the R¹O—(C═O)—CH₂—CO group, Xaa² being valine and R¹ the ethyl group, so as to confer on said substrate a low affinity with respect to proteolytic enzymes.

7. The method of claim 1, wherein in the proteolytic enzyme substrate Q² comprises a —(C═O)ₘ—(O)ₙ— group linked to rhodamine₁₁₀, in which n=0 or 1, and m=0 or 1.

8. The method of claim 7, wherein in the proteolytic enzyme substrate n=0 and m=1.

9. The method of claim 7, wherein in the proteolytic enzyme substrate wherein Q² is the acetyl group.

10. The method of claim 7, wherein in the proteolytic enzyme substrate Q² is the 3-methylbutanoyl group.

11. Method for measuring the activity of proteolytic enzymes of a blood sample, comprising:
providing a blood sample which contains proteolytic enzymes and which may contain a glycosaminoglycan;
providing a proteolytic enzyme substrate capable of providing a signal when said proteolytic enzymes react with said substrate;
bringing said blood sample into contact with said proteolytic enzyme substrate so as to allow said proteolytic enzymes to react with said substrate in order to provide a signal representative of the activity of the proteolytic enzymes in said sample;
using a proteolytic enzyme substrate according to claim 1, so as to be able both to allow the glycosaminoglycan to inhibit the coagulation of said blood sample and to measure the activity of the proteolytic enzymes, via which the signal measured is representative of the activity of only the proteolytic enzymes capable of bringing about coagulation.

12. Measuring method according to claim 11, further comprising, providing an activating reagent which makes it possible to induce the generation of proteolytic enzymes, and bringing said activating reagent into contact with said blood sample and said proteolytic enzyme substrate.

13. Measuring method according to claim 12, further comprising, electing said activating reagent from the group consisting of tissue factor, phospholipids, thromboplastin, kaolin, ellagic acid, collagen, adenosine diphosphate, arachidonic acid, a thrombin receptor-activating peptide, or a combination thereof.

14. Measuring method according to claim 11, further comprising, providing an initiating reagent for initiating the proteolytic enzyme reactions, and bringing said initiating reagent into contact with said blood sample and said proteolytic enzyme substrate.

15. Measuring method according to claim 14, wherein said initiating reagent is calcium.

16. Measuring method according to claim 11, further comprising, bringing said blood sample into contact with said proteolytic enzyme substrate at a substrate concentration of between 50×10⁻⁶ and 1000×10⁻⁶ mol.L⁻¹.

17. A kit for measuring the activity of proteolytic enzymes of a blood sample, for implementing the method according to claim 11, said blood sample containing a glycosaminoglycan, said glycosaminoglycan inhibiting the coagulation of said blood sample, via which the anticoagulant capacity of the glycosaminoglycan is not disrupted, the kit comprising:
an activating reagent for inducing the generation of proteolytic enzymes;
a proteolytic enzyme substrate of general formula: Q¹-Xaa²-Xaa¹-rhodamine₁₁₀-Q², in which:
Xaa¹ and Xaa² are amino acids; and
Q² is a group comprising an alkyl group; and
an initiating reagent for initiating the enzymatic reactions.

18. A measuring kit according to claim 17, wherein said proteolytic enzyme substrate is mixed with said initiating reagent.

19. A measuring kit according to claim 17, wherein said activating reagent is mixed with said initiating reagent.
